(19) United States
(12) Patent Application Publication
(10) Pub. No.: US 2011/0190687 A1
(43) Pub. Date: Aug. 4, 2011

(54) APPLICATOR HAVING IMPROVED SURFACE ELEMENTS

(76) Inventors: Nancy Deters SLAYTON, Springfield, OH (US); Eric Mitchell Schickli, Cincinnati, OH (US); David Andrew Dalton, Mason, OH (US); Reginald Edward Crutcher, II, Cincinnati, OH (US)

(21) Appl. No.: 13/013,931

(22) Filed: Jan. 26, 2011

Related U.S. Application Data
(60) Provisional application No. 61/299,620, filed on Jan. 29, 2010.

(51) Int. Cl. A61F 13/32

(52) U.S. Cl. 604/15

(57) ABSTRACT

An applicator for a feminine hygiene device. The applicator includes an insertion member that can have an outer surface, an insertion end, a withdrawal end opposite the insertion end, a barrel region adapted to house the feminine hygiene device, and a grip region disposed proximate the withdrawal end. In certain embodiments, the grip region can include one or more three-dimensional surface elements protruding from the grip region. In addition, the barrel region can include one or more two-dimensional surface elements.

### CROSS REFERENCE TO RELATED APPLICATION

This application claims the benefit of U.S. Provisional Application No. 61/299,620 (aka 11594P), filed Jan. 29, 2010.

### FIELD OF THE INVENTION

The present invention relates to an improved applicator for feminine hygiene devices, and more particularly, to an improved applicator for feminine hygiene devices having improved surface elements.

### BACKGROUND OF THE INVENTION

Feminine hygiene devices, such as tampons and pessaries, are generally used by women within the vagina for feminine needs, such as, e.g., to absorb menstrual or other body exudates, for pelvic support, and/or for other feminine needs. Such feminine products can be inserted into the vagina digitally, such as, e.g., by using a finger, or can be inserted into the vagina by using an applicator.

Applicators typically can comprise an insertion member and a plunger. The material to be expelled from the applicator, such as an absorbent tampon or pessary, can be positioned within the insertion member. The insertion member can have a first end for insertion of the material and a second end for receipt of the plunger. To use the applicator, the consumer can grasp the insertion member, position the first end appropriately, such as, e.g., into the body, and move the plunger in the insertion member towards the first end to insert the material. Some applicators can also include a fingergrip configuration that is located on the insertion member, which can allow the consumer to more securely hold the applicator during insertion of a material into the body cavity.

Various fingergrip configurations have been proposed to facilitate the handling of the applicator and to improve the insertion experience. For example, certain applicators can include a straight fingergrip configuration, wherein the fingergrip is not indented from the remainder of the insertion member. In some instances, a straight fingergrip configuration can also include one or more gripping structures disposed proximate the withdrawal end of the insertion member, that can, for example, reduce finger slippage and provide a more secure grip. Alternatively, other applicators can include a fingergrip configuration having a reduced diameter compared to the remainder of the insertion member, such as, e.g., one or more indentation regions or a rearwardly decreasing diameter that can, for example, provide one or more tapers or shoulders demarcating the gripping region from the remainder of the insertion member. Generally, such applicators can contain one or more gripping structures disposed within the fingergrip area. Such gripping structures are typically discrete elements, such as, for example, ribs or rings disposed about the perimeter of the grip, or circles, or other discrete elements.

Although many different types of applicators for feminine hygiene devices have been previously described, currently available configurations are not yet optimized to facilitate applicator handling and insertion experience. As such, there remains a need for an improved applicator having a superior grip configuration and improved perception of comfort.

### SUMMARY OF THE INVENTION

An applicator for a feminine hygiene device is provided. The applicator includes an insertion member. The insertion member can have an outer surface, an insertion end, a withdrawal end opposite the insertion end, and a barrel region adapted to house the feminine hygiene device. In addition, the insertion member can have grip region disposed proximate the withdrawal end. In certain embodiments, the grip region can include one or more three-dimensional surface elements protruding from the grip region. In addition, in certain embodiments, the barrel region can include one or more two-dimensional surface elements.

An applicator for a feminine hygiene device is also provided. The applicator includes an insertion member. The insertion member can have an outer surface, an insertion end, a withdrawal end opposite the insertion end, and a barrel region adapted to house the feminine hygiene device. In addition, the insertion member can have an indentation region extending inwardly from the outer surface and disposed between the barrel region and withdrawal end, and a shoulder region disposed between the barrel region and the indentation region. In certain embodiments, the indentation region can include one or more three-dimensional surface elements protruding from the indentation region, the one or more three-dimensional surface elements extending from the indentation region over the shoulder region. In addition, in certain embodiments, the barrel region can include one or more two-dimensional surface elements.

In addition, an applicator for a feminine hygiene device is provided. The applicator includes an insertion member. The insertion member can have an outer surface, an insertion end, a withdrawal end opposite the insertion end, and a barrel region adapted to house the feminine hygiene device. In addition, the insertion member can have an indentation region extending inwardly from the outer surface and disposed between the barrel region and withdrawal end, and a shoulder region disposed between the barrel region and the indentation region. In certain embodiments, the indentation region can include a plurality of three-dimensional surface elements protruding from the indentation region, the three-dimensional surface elements extending from the indentation region over at least a portion of the shoulder region. In addition, in certain embodiments, the barrel region can include one or more two-dimensional surface elements. In addition, in certain embodiments, the three-dimensional surface elements and the two-dimensional surface elements can have the same shape.

### BRIEF DESCRIPTION OF THE DRAWINGS

FIG. 1 is a side view of a feminine hygiene device applicator.

FIG. 2 is a side view of a feminine hygiene device applicator.

FIG. 3 is a side view of a feminine hygiene device applicator.

### DETAILED DESCRIPTION OF THE INVENTION

Improved feminine hygiene device applicators are provided, that can, for example, provide a consumer with an improved grip, improved insertion comfort, and/or improved perception of comfort, stability, and/or of not slipping during use of the applicator. In certain embodiments, the feminine hygiene device applicator can comprise improved surface elements. Such surface elements may generally include, for example, three-dimensional surface elements and/or two-dimensional surface elements. In certain embodiments, the surface elements can include three-dimensional surface elements having improved configurations of three-dimensional surface elements that protrude from the grip region of the feminine device applicator to provide a gripping surface. In certain embodiments, the three-dimensional surface elements can include one or more overlapping three-dimensional surface elements. In addition, in certain embodiments, the surface elements can include barrel elements having improved configurations of visible two-dimensional surface elements that can extend longitudinally along the length of the barrel, such as, for example, from the grip region of the feminine device applicator towards the insertion end of the feminine device applicator. In certain embodiments, the two-dimensional surface elements will not extend to the petal region.

In addition, in certain embodiments, the feminine hygiene device applicator can include an insertion member having an indented fingergrip region, and the insertion member can include one or more shoulder regions provided between the indented fingergrip region and the remainder of the insertion member. As described herein, the indented fingergrip region can also include one or more three-dimensional surface elements that can extend beyond the fingergrip region over at least a portion of the shoulder region, or, in certain embodiments, over the shoulder region and over at least a portion of the remainder of the insertion member.

Such applicator configurations can provide an improved gripping experience and can also provide the perception of a slimmer, more comfortable applicator. In addition, it has been discovered that the improved surface elements can impact consumer rating and perception of insertion comfort and of a non-slip grip.

As used herein, the term “feminine hygiene device” includes absorbent articles useful for feminine needs, such as articles that typically can be intended for feminine use internally, such as, e.g., within a user's vagina. Internal feminine hygiene devices can include, for example, tampons and pessaries.

As used herein, the term “tampon” refers to any type of absorbent structure that can be inserted into the vaginal canal or other body cavity, such as, e.g., for the absorption of fluid, to aid in wound healing, and/or for the delivery of materials, such as moisture or active materials such as medicaments.

As used herein, the term “pessary” refers to any type of substantially non-absorbent structure for the purpose of reducing urine leakage and/or supporting a prolapsed uterus and/or bladder. Such pessaries can have any variety of shapes and sizes including cylinder, ovate, spherical, tubular, annual rings, “U” shaped, cup shaped, rings, cubes or donut shaped, and can function in any suitable manner, such as, e.g., by direct application of support, lever force, expansion of the device by selection of material, and/or by inflation of the device.

As used herein, the term “vaginal canal” refers to the internal genitalia of the human female in the pudendal region of the body. The terms “vaginal canal” or “within the vagina” as used herein are intended to refer to the space located between the introitus of the vagina (sometimes referred to as the sphincter of the vagina) and the cervix. As used herein, “applicator” refers to a device or implement that facilitates the insertion of a feminine hygiene device, such as, e.g., a tampon or pessary, into an external orifice of a mammal. Exemplary applicators include telescoping, tube and plunger, and compact applicators.

As used herein, the term “insertion end” refers to the portion of the tampon or applicator including the end that is intended to enter the vaginal canal first when inserting the tampon or applicator into the vaginal canal.

As used herein, the term “withdrawal end” refers to the portion of the applicator opposite the insertion end including the end is intended to exit the vaginal canal first when the applicator is removed from the vagina.

As used herein, the term “barrel region” refers to the portion of the applicator adapted to house the feminine hygiene device. In certain embodiments, the barrel region includes the region of the applicator having the largest diameter.

As used herein, the term “grip region” refers to the portion of the applicator that adapted to provide a gripping surface that can facilitate grasping and/or holding of the applicator. In certain embodiments, the grip region can be an indentation region.

As used herein, the term “indentation region” refers to the region of the insertion member having the smallest diameter. The indentation region is not located on the plunger.

As used herein, the term “shoulder region” refers to the upper region of the surface providing the slope or angle from the barrel region to the indentation region.

As used herein, the term “surface elements” refers to three-dimensional or two-dimensional elements disposed on the feminine device applicator surface. Surface elements are “visually perceptible” to a user viewing the feminine device applicator surface. By “visually perceptible” is meant that a human viewer can visually discern the surface element with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 0.25 meters.

As used herein, the term “three-dimensional surface element” refers to a surface element that is raised or depressed as compared to the feminine device applicator surface such that the element is visually and tactilely perceptible to a user viewing and grasping the three-dimensional surface element.

As used herein, the term “two-dimensional surface element” refers to a surface element that is visually perceptible to a user viewing the feminine device applicator, but that is not tactilely perceptible to a user viewing and grasping the feminine device applicator. In addition, in certain embodiments, the two-dimensional surface element can be a microtexture.

As used herein, the term “gloss” refers to an area of a feminine device applicator that has a gloss measurement of greater than about 200 gloss units as measured using ASTM D2457-08 Standard Test Method for Specular Gloss of Plastic Films and Solid Plastics at a 60 degree angle, with samples attached in a planar configuration to a flat optical black plate as a backing. As used herein, the term “matte” refers to an area of a feminine device applicator that has a gloss measurement of less than about 70 gloss units as measured using ASTM D2457-08 Standard Test Method for Specular Gloss of Plastic Films and Solid Plastics at a 60 degree angle, with samples attached in a planar configuration to a flat optical black plate as a backing.

FIG. 1 shows one embodiment of an applicator 10. The applicator 10 comprises an insertion member 20 and a plunger 40. The insertion member 20 has an insertion end 21 and a withdrawal end 22 opposite the insertion end 21. The insertion member 20 can also include a barrel region 50 adapted to contain a feminine hygiene device such as, e.g., a tampon, and a grip region 30 that can, in certain embodiments, be an indentation region provided opposite the insertion end 21, such as, e.g., proximal to the withdrawal end 22. In addition, in certain embodiments, such as, for example, when the grip region 24 is an indentation region, the grip region 24 can be demarcated from the barrel region 50 of the insertion member 20, such as, e.g., by one or more shoulder regions 26. As shown in FIG. 1, grip region 24 can, in certain embodiments, be disposed continuously about the circumference of the insertion member 20. In addition, the grip region 24 can comprise three-dimensional surface elements 27 that can protrude outward from the grip region 24. As shown in FIG. 1, in certain embodiments, one or more three-dimensional surface elements 27 can extend from the grip region 24 over at least a portion of one or more shoulder regions 26.

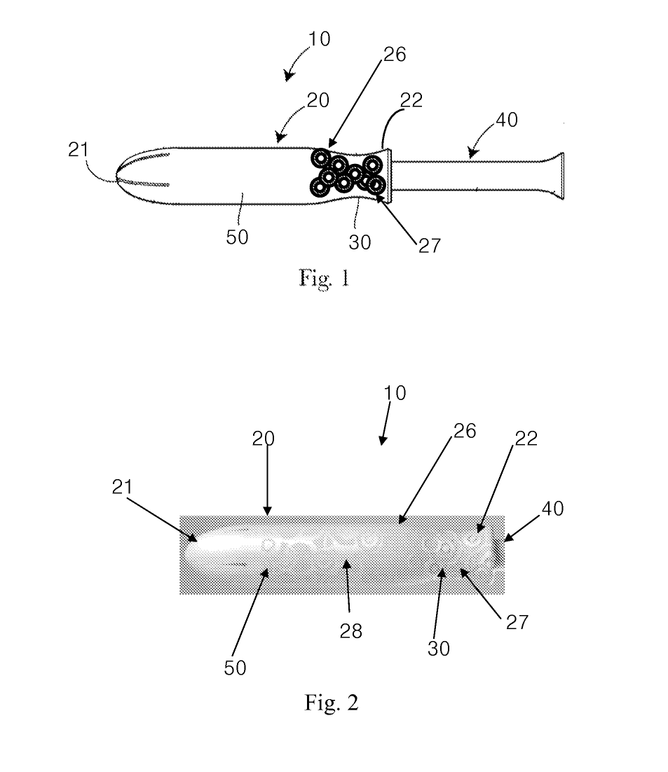

FIG. 2 shows one embodiment of an applicator 10. The applicator 10 comprises an insertion member 20 and a plunger 40. The insertion member 20 has an insertion end 21 and a withdrawal end 22 opposite the insertion end 21. The insertion member 20 can also include a barrel region 50 adapted to contain a feminine hygiene device such as, e.g., a tampon, and a grip region 30 that can be an indentation region provided opposite the insertion end 21, such as, e.g., proximal to the withdrawal end 22. In addition, in certain embodiments, the grip region 24 can be demarcated from the barrel region 50 of the insertion member 20, such as, e.g., by one or more shoulder regions 26. As shown in FIG. 2, grip region 24 can, in certain embodiments, be disposed continuously about the circumference of the insertion member 20. In addition, the grip region 24 can comprise three-dimensional surface elements 27 that can protrude outward from the grip region 24. As shown in FIG. 2, in certain embodiments, one or more three-dimensional surface elements 27 can extend from the grip region 24 over at least a portion of one or more shoulder regions 26.

In addition, the feminine hygiene device applicator can, in certain embodiments, include two-dimensional surface elements 28 provided on the barrel and/or plunger of the applicator. As shown in FIG. 2, the two-dimensional surface elements 28 can be in the same shape and/or configuration as the three-dimensional surface elements 27. For example, in this embodiment, both two-dimensional elements 28 and three-dimensional surface elements 27 are circles provided in an overlapping configuration, that is, the circles overlap adjacent circles.

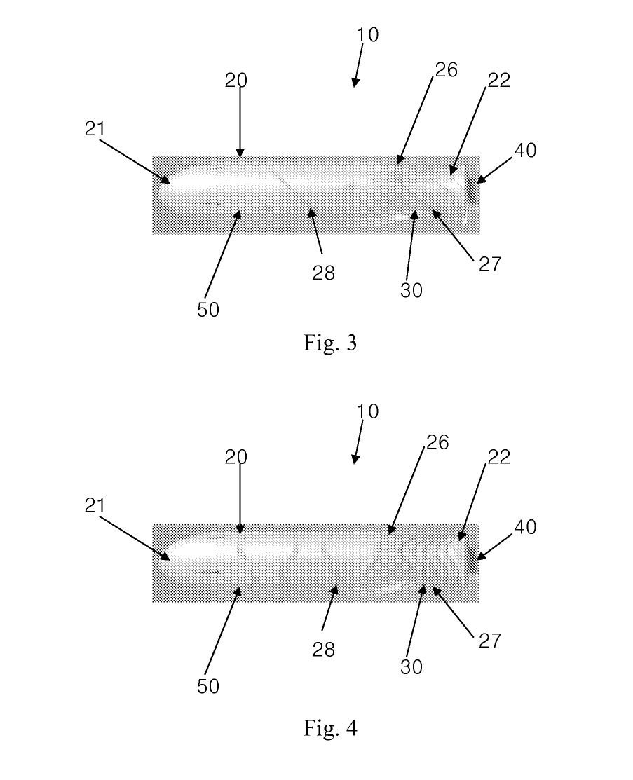

FIG. 3 shows one embodiment of an applicator 10. The applicator 10 comprises an insertion member 20 and a plunger 40. The insertion member 20 has an insertion end 21 and a withdrawal end 22 opposite the insertion end 21. The insertion member 20 can also include a barrel region 50 adapted to contain a feminine hygiene device such as, e.g., a tampon, and a grip region 30 that can be an indentation region provided opposite the insertion end 21, such as, e.g., proximal to the withdrawal end 22. As shown in FIG. 3, the grip region 24 protrudes outwardly from an outer surface of the insertion member 20. In addition, in certain embodiments, the grip region 24 can be demarcated from the barrel region 50 of the insertion member 20, such as, e.g., by one or more shoulder regions 26. As shown in FIG. 3, grip region 24 can, in certain embodiments, be disposed continuously about the circumference of the insertion member 20. In addition, the grip region 24 can comprise three-dimensional surface elements 27 that can protrude outward from the grip region 24. As shown in FIG. 3, in certain embodiments, one or more three-dimensional surface elements 27 can extend from the grip region 24 over at least a portion of one or more shoulder regions 26. As shown in FIG. 3, in certain embodiments, three-dimensional surface elements 27 can be provided in a pattern, wherein the pattern can extend from the grip region 24 over one or more shoulder regions 26, and over at least a portion of barrel region 23.

In addition, the feminine hygiene device applicator can, in certain embodiments, include two-dimensional surface elements 28 provided on the barrel and/or plunger of the applicator. As shown in FIG. 3, the two-dimensional surface elements 28 can be in the same shape and/or configuration as the three-dimensional surface elements 27. For example, in this embodiment, both two-dimensional elements 28 and three-dimensional surface elements 27 are combinations of lines and stars provided in a connecting configuration, that is, the lines connect to the stars.

In certain embodiments, the feminine hygiene device applicator includes three-dimensional surface elements proximate the grip region of the applicator and two-dimensional surface elements proximate the barrel region of the applicator. In certain embodiments, the three-dimensional surface elements and the two-dimensional surface elements include the same shape. In addition, in certain embodiments, the three-dimensional surface elements and the two-dimensional surface elements are in the same pattern and/or configuration, such as, for example, overlapping, connecting, or discrete. In certain embodiments, the three-dimensional surface elements and the two-dimensional surface elements can have overlapping elements. Alternatively, the three-dimensional surface elements can have overlapping elements and the two-dimensional surface elements can have discrete elements.

In certain embodiments, the two-dimensional surface elements can be provided in a longitudinal direction, that is, the two-dimensional surface elements or groups of elements can be generally provided along the length of applicator, such as, for example, substantially parallel with the longitudinal axis of the feminine hygiene device applicator. In certain embodiments, the two-dimensional surface elements can be considered discrete and separate from one another, such as, for example, where the two-dimensional surface elements are separated from one another by a surface of the feminine hygiene device applicator that does not include two-dimensional surface elements. In certain embodiments, the feminine hygiene device applicator can have a gloss surface and the two-dimensional surface elements can have a matte appearance, or the reverse, as long as the two-dimensional surface elements are visually perceptible to a user viewing the applicator.

As set forth herein, the three-dimensional surface elements can be provided in the grip region, such as, for example, a grip region that is an indentation region, and over at least a portion of a shoulder region adjacent the grip region and/or over the shoulder region onto at least a portion of the barrel region. Generally, in embodiments where the grip region includes an indentation region, the shoulder region can refer to the upper region of the slope or angle from the barrel region to the indentation region, and in certain embodiments, may be further defined as the upper half of the surface having a slope or angle beginning at the barrel region and ending at the indentation region, such as, e.g., the upper half closest to the barrel region of the applicator. In addition, in certain embodiments, the shoulder region can be further defined as the upper third or upper quarter of the surface having a slope or angle beginning at the barrel region and ending at the indentation region, such as, e.g., the upper third or upper quarter closest to the barrel region of the applicator. In certain embodiments, the three-dimensional surface elements can extend only over the shoulder region. Alternatively, in certain embodiments, the three-dimensional surface elements can also extend over the portion of the barrel region closest to the indentation region, such as, e.g, over about 10%, over about 20%, over about 30%, or over any other suitable amount of the length of the barrel region closest to the indention region. As such, in certain embodiments, the three-dimensional surface elements do not extend to the insertion end of the applicator. In addition, in certain embodiments, the three-dimensional surface elements can be provided on the barrel portion in a region where the diameter is not increasing toward the insertion end, and/or can be provided on the barrel region up to or over the maximum diameter point of the applicator.

The three-dimensional surface elements can be provided in any suitable configuration, such as, e.g., overlapping configurations, longitudinal formations, waves, swirls, a substantially contiguous pattern of individual elements, a substantially contiguous pattern of joined elements, or any other suitable configuration. In certain embodiments, the application can include at least one elongated gripping formation provided within indentation region in a direction generally parallel to the longitudinal axis (L) of the applicator, wherein the at least one elongated gripping formation includes one or more three-dimensional surface elements or a series of overlapping surface elements that can extend from the indentation region over at least a portion of a shoulder region of the applicator.

In addition, the grip region can comprise any suitable shape and/or configuration that can facilitates grasping and/or holding of the applicator. For example, in certain embodiments, the grip region can be an indention region that can be a shape and/or configuration suitable for positioning one or more of a user's fingers within the indention region. For example, in certain embodiments, the grip region can be disposed continuously about the circumference of the insertion member. Alternatively, the grip region can include one or more discrete elements. The grip region can have any suitable shape and/or cross-section, such as, e.g., circular, oval, elliptical, or a cross-section having a non-arcuate perimeter, such as, e.g., a square, rectangular, triangular, polygonal, flattened, or other suitable cross-sectional shape. In certain embodiments, the grip region can have a perimeter wherein a portion of the perimeter is arcuate and wherein a portion of the perimeter is non-arcuate, such as, e.g., an grip region with one or more curved sides and one or more flattened sides.

The insertion member can be constructed from any suitable material. Suitable materials include, for example, paper, paperboard, cardboard, cellulose, such as, e.g., molded cellulose, or any combinations thereof, polyethylene, polypropylene, polybutylene, polystyrene, polyvinylchloride, polyacrylate, polymethacrylate, polyacrylonitrile, polyacrylamide, polyamide, nylon, polyimide, polyester, polycarbonate, polylactic acid, poly hydroxyalkanoate, ethylene vinyl acetate, polyurethane, silicone, derivatives thereof, copolymers thereof, mixtures thereof, or any suitable smooth plastic material. Examples of suitable materials are disclosed in, e.g., U.S. Pat. Nos. 5,346,468 and 5,558,631. In certain embodiments, additives can be included in the material to alter or enhance certain material properties. Suitable additives include, for example, mold release agents, slip agents, surface energy modifiers, pearlescent agents, and/or any other suitable additives. In certain embodiments, the insertion member can be coated with a substance to give it a high slip characteristic, such as, e.g., with wax, polyethylene, a combination of wax and polyethylene, cellophane, clay, mica, and other lubricants that can facilitate comfortable insertion. Alternatively, or in addition, the insertion member can include a textured surface. Texture can be provided in any suitable manner, such as, e.g., by designing texture into or adding texture to the insertion member.

In certain embodiments, the insertion member can be in the form of a spirally wound, convolutely wound or longitudinally seamed hollow tube, which can be formed from paper, paperboard, cardboard or a combination thereof. The insertion member can have one or more walls of any suitable thickness. In certain embodiments, the one or more walls can have a predetermined thickness of from about 0.1 millimeters to about 0.7 millimeter. The wall can be constructed from a single ply of material or can be formed from two or more plies that are bonded together, such as, e.g., to form a laminate. When two or more plies are utilized, some or all of the plies can be spirally wound, convolutely wound or longitudinally seamed to form an elongated cylinder. For example, in certain embodiments the wall can be constructed using a smooth thin ply of material on the outside or exterior surface that surrounds a coarser and possibly thicker ply. In embodiments where the wall contains at least three plies, the middle ply can be the thicker ply and the interior and exterior plies can be smooth and/or slippery to facilitate expulsion of the tampon and to facilitate insertion of the insertion member. The wall can contain one to four plies, although more plies can be utilized if desired.

The plies can be held together in any suitable manner, such as, e.g., by one or more adhesives, such as glue, by heat, by pressure, by ultrasonics, or by any other suitable manner for holding the plies together. The adhesive can be either water-soluble or water-insoluble. In certain embodiments, a water-soluble adhesive can be used such that the wall will quickly break apart when it is immersed in water, such as, e.g., by flushing the insertion member down a toilet.

Alternatively, the material can be overlapped into a tubular configuration, such as, for example, by spirally or convolutely winding the insertion member into a cylindrical tube. In the case of other tube construction methods such as fiber or plastic molding, or integral tube forming (e.g., thermoforming plastic) no seams may be present and the corrugations could optionally be formed as part of the tube molding or forming process.

As set forth herein, the insertion member includes an indentation region having a plurality of three-dimensional surface elements, such as, e.g., projections, rings, ridges, ribs, embossments, depressions, grooves, and/or other gripping structures. The three-dimensional surface elements can be provided in any suitable manner, such as, e.g., by the addition of material, and/or by impressing, such as, e.g., by embossing, or compressing the surfaces. In certain embodiments, the indentation region can include one or more flattened sides and/or one or more spaces for a decorative marking or a character, such as, e.g., an embossed and/or printed marking or character. In addition, or alternatively, the surfaces of the indentation region can include a material that can provide a frictional resistance for the user's fingers during the insertion of the tampon applicator into the body. Suitable materials that can provide friction include, for example, abrasive materials, high wet coefficient of friction materials, pressure sensitive adhesives, or any combinations thereof.

The three-dimensional surface elements can have any suitable dimensions. In certain embodiments, the three-dimensional surface elements can be elongated, such as, e.g., having a length greater than the width. In addition or alternatively, the three-dimensional surface elements can have a height extending outward from the surface of the insertion member. In certain embodiments, the three-dimensional surface elements can have a height greater than about 0.1 mm, such as, e.g., a height from about 0.1 mm to about 0.8 mm, such as, e.g., a height from about 0.1 mm to about 0.6 mm, such as, e.g., a height from about 0.15 mm to about 0.5 mm, such as, e.g., a height from about 0.2 mm to about 0.4 mm, such as, e.g., a height from about 0.2 mm to about 0.3 mm, or any other suitable height.

In certain embodiments, the three-dimensional surface elements can have varying heights along the length of the applicator. For example, in certain embodiments, the three-dimensional surface elements can be higher in the indentation region and lower on the shoulder region and/or barrel region. Alternatively, in certain embodiments, the three-dimensional surface elements can be higher on the shoulder and/or barrel and lower in the indentation region.

The three-dimensional surface elements can be provided in the grip region in any suitable location. In certain embodiments, the three-dimensional surface elements can form a design, such as, e.g., a pattern. The three-dimensional surface elements can be arranged to form a perceived pattern, such as, e.g., a line, a broken line, waves, swirls, a ring, and/or a broken ring. For example, in certain embodiments, a plurality of three-dimensional surface elements can be arranged to form a series of waves, such as, e.g., one or more waves having a crest provided within the indentation region and a trough provided over the shoulder region and/or onto the barrel region. Alternatively, in certain embodiments, a plurality of three-dimensional surface elements can be arranged to form a series of overlapping elements, such as, for example, overlapping circles that continue from the indentation region onto the shoulder region of the feminine hygiene device applicator.

The two-dimensional surface elements can be provided in any suitable manner. For example, in certain embodiments, the two-dimensional surface elements can be provided by etching, molding, or otherwise texturing the applicator, such as, for example, by microtexturing the two-dimensional surface elements onto the surface of the feminine hygiene device applicator. In certain embodiments, the feminine hygiene device applicator can be molded with a specified finish, such as, for example, a gloss finish, and the two-dimensional surface elements can be etched into the surface of the applicator to provide a matte finish appearance. Alternatively, in certain embodiments, the feminine hygiene device applicator can be injection molded using a mold with gloss areas and matte areas to provide the visually perceptible two-dimensional surface elements described herein.

The two-dimensional surface elements can be provided in any suitable location on the barrel region. In certain embodiments, the two-dimensional elements can be provided adjacent to the three-dimensional surface elements. Alternatively, the two-dimensional surface elements can be spaced from the three-dimensional surface elements, such as, for example, provided closer to the insertion end of the applicator as compared to the three-dimensional surface elements. In certain embodiments, the two-dimensional surface elements do not extend to the petals of the applicator.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An applicator for a feminine hygiene device comprising:
an insertion member;
the insertion member having an outer surface, an insertion end, a withdrawal end opposite the insertion end, and a barrel region adapted to house the feminine hygiene device, the insertion member having a grip region;
the grip region comprising one or more three-dimensional surface elements protruding from the grip region, the one or more three-dimensional surface elements extending from the grip region over at least a portion of the shoulder region; and
the barrel region comprising one or more two-dimensional surface elements.

2. The applicator of claim 1, wherein the grip region is disposed continuously about the circumference of the insertion member.

3. The applicator of claim 1, wherein the grip region comprises one or more overlapping three-dimensional surface elements.

4. The applicator of claim 1, wherein the grip region includes an indentation region.

5. The applicator of claim 1, wherein the one or more three-dimensional surface elements are a series of overlapping three-dimensional surface elements providing a pattern originating in the indentation region and terminating in the shoulder region.

6. The applicator of claim 1, wherein the two-dimensional surface elements comprise a micro-texture.

7. The applicator of claim 1, wherein the outer surface has a gloss finish and the two-dimensional surface elements have a matte finish or vice versa.

8. The applicator of claim 1, wherein the applicator is a tampon applicator.

9. An applicator for a feminine hygiene device comprising:
an insertion member;
the insertion member having an outer surface, an insertion end, a withdrawal end opposite the insertion end, and a barrel region adapted to house the feminine hygiene device, the insertion member having an indentation region extending inwardly from the outer surface and disposed between the barrel region and withdrawal end, the insertion member further having a shoulder region disposed between the barrel region and the indentation region;
the indentation region comprising one or more three-dimensional surface elements protruding from the indentation region, the one or more three-dimensional surface elements extending from the indentation region over the shoulder region;
the barrel region comprising one or more two-dimensional surface elements.

10. The applicator of claim 9, wherein the indentation region is disposed continuously about the circumference of the insertion member.

11. The applicator of claim 9, wherein the indentation region comprises one or more overlapping three-dimensional surface elements.

12. The applicator of claim 9, wherein the one or more three-dimensional surface elements protrude outwardly from the indentation region.

13. The applicator of claim 9, wherein the one or more three-dimensional surface elements are a series of overlapping three-dimensional surface elements providing a pattern originating in the indentation region and terminating in the barrel region.

14. The applicator of claim 9, wherein the two-dimensional surface elements comprise a micro-texture.

15. The applicator of claim 9, wherein the two-dimensional surface elements do not extend to the petals.

16. The applicator of claim 9, wherein the one or more three-dimensional surface elements extend over less than about 30% of the length of the barrel region.

17. An applicator for a feminine hygiene device comprising:
an insertion member;
the insertion member having an outer surface, an insertion end, a withdrawal end opposite the insertion end, and a barrel region adapted to house the feminine hygiene device, the insertion member having an indentation region extending inwardly from the outer surface and disposed between the barrel region and withdrawal end, the insertion member further having a shoulder region disposed between the barrel region and the indentation region;
the indentation region comprising a plurality of three-dimensional surface elements protruding from the indentation region, the three-dimensional surface elements extending from the indentation region over at least a portion of the shoulder region;
the barrel region comprising a plurality of two-dimensional surface elements; and
wherein the two-dimensional surface elements and the three-dimensional surface elements comprise the same shape.

18. The applicator of claim 17, wherein the indentation region is disposed continuously about the circumference of the insertion member.

19. The applicator of claim 17, wherein the two-dimensional surface elements are one or more discrete elements.

20. The applicator of claim 17, wherein one or more of the two-dimensional surface elements are overlapping.
